(19) United States
(12) Patent Application Publication
(10) Pub. No.: US 2017/0333158 A1
(43) Pub. Date: Nov. 23, 2017

(54) FIBERGLASS DENTAL CROWNS

(71) Applicant: Figaro Crowns Inc., Woodbury, MN (US)

(72) Inventor: Andy Mancini, Woodbury, MN (US)

(73) Assignee: Figaro Crowns Inc., Woodbury, MN (US)

(21) Appl. No.: 15/593,526

(22) Filed: May 12, 2017

Related U.S. Application Data
(60) Provisional application No. 62/338,809, filed on May 19, 2016.

(51) Int. Cl. A61C 5/73; A61C 5/77; A61K 6/00; A61K 6/087

(52) U.S. Cl. CPC A61C 5/73; A61K 6/087; A61C 5/77; A61K 6/0091; A61K 6/0094

(57) ABSTRACT

A dental crown composed of fiber mesh sheets of fiberglass, aramid, carbon or quartz fibers embedded within dentally acceptable resin. The combination of both materials synergistically add unsurpassed strength and enhanced cosmetic value to the dental crown for a much lower price due to the cheaper costs of the material and manufacturing process.

### CROSS-REFERENCE TO RELATED APPLICATIONS

This application claims priority to U.S. Provisional patent application 62/338,809 filed on May 19, 2016, the disclosure of which is incorporated herein by reference as permitted.

### STATEMENT REGARDING FEDERALLY SPONSORED RESEARCH

Not Applicable

### BACKGROUND OF THE INVENTION

### Field of the Invention

A dental crown wherein the crown is made with a fiberglass or quartz fiber embedded with cosmetic resin composite.

### Description of the Related Art

Dental crowns may be made of molded zirconia but are quite expensive and their pricing limits its acceptable throughout the general population. Stainless steel crowns are less expensive but require the use of a protective “unnatural” color crown restoration for primary teeth. The stainless steel construction has a major problem, that the “silver” color of the steel crown is not acceptable, and the issue with the use of a zirconia cosmetic crown involves beyond the necessary thus excessive grinding of tooth structure to compensate for the lack of flexibility thus leading to some retention, adaptability and strength problems.

There is a need for an excellent dental crown that is lower cost, in particular for pediatric dentistry where cost may be even more of an issue and baby teeth would eventually fall out.

### BRIEF SUMMARY OF THE INVENTION

The invention provides a dental crown that is an inexpensive and affordable solution for restorations in pediatric and adult dentistry utilizing crowns, with added cosmetic value not allowed with the stainless steel crowns and not affordable with zirconia type crowns. The invention utilizes either fiberglass or quartz filaments/fibers imbedded with an outer cosmetic composite resin material embodying the crown has a similar structure observed on fiberglass dental posts already widely used in dentistry for endodontic/restorative purposes for decades. The strength and bio-compatibility with a degree of flexibility is much closer to tooth structure then stainless steel and zirconia crowns.

### BRIEF DESCRIPTION OF THE SEVERAL VIEWS OF THE DRAWINGS

FIG. 1 is a view of a dental crown of the invention in perspective view showing one of the reinforcing layers in a top view;

FIG. 2 is a view of a dental crown of the invention in perspective view showing a second reinforcing layer in a top view;

FIG. 3 is a view of a dental crown of the invention in perspective view showing the third reinforcing layer in a top view;

FIG. 4 is a view of the reinforcing layers of the invention in a top view;

FIG. 5 is a perspective view of a typical tooth ready for a dental crown of the invention;

FIG. 6 is a perspective view of the dental crown of the invention secured to the tooth of FIG. 5; and

FIG. 7 is a cross-sectional view of a dental crown of the invention affixed to a living tooth in a patient.

### DETAILED DESCRIPTION OF THE INVENTION

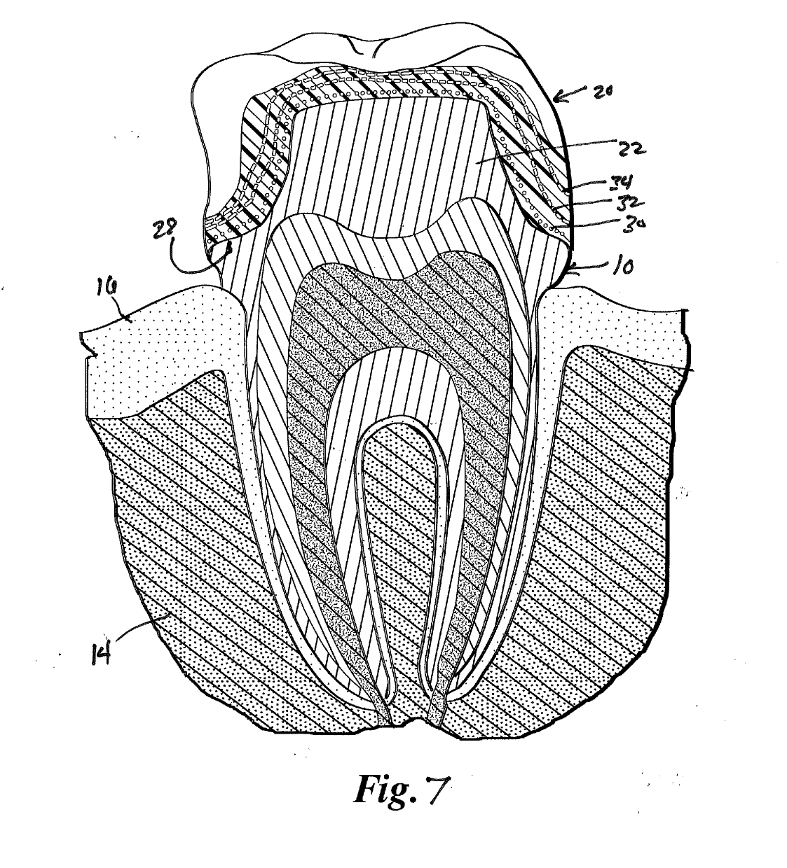

The invention as shown in the Figures is a dental crown using fiberglass, aramid, carbon or quartz filaments/fibers imbedded with an outer cosmetic composite resin material. As shown in FIG. 7, a natural tooth 10 includes roots 12 which are secured in alveolar bone 14. The gum 16 is on top of bone 14 and surrounds tooth 10. The exterior biting surfaces of the tooth 10 are enamel and in a dental crown procedure the tooth is ground down to form a support onto which the dental crown 20 may be affixed. In some cases, the existing tooth does not have sufficient structure to hold the restorative dental crown 20 in place and dental posts are installed which form the basis of the material to which the dental crown 20 is affixed.

As shown in FIG. 7, the dental crown 20 of the invention is affixed on top of the defined dentin 22 although it would be the same as if a dental post was in place. The dental crown 20 is a cup-shaped cap having a closed end 24 forming the occlusal table and sides which terminate in what is called the gingival extremity 26. The interior 28 of the dental crown 20 is matched to mate with either the prepared tooth dentin 22 or a dental post (not shown). The securement of a dental crown to the tooth or post is well known in the art. The occlusal anatomy of the dental crown in general conforms in the location of the sulcus groove, supplemental occlusal grooves, the cusps and the cuspal planes with occlusal anatomy of the tooth. The topography of the dental crown 20 of the invention externally is that of any acceptable crown for the tooth involved.

The fiberglass or quartz fiber containing dental crowns of the invention may be made with the same materials currently used in FiberKleer® Posts from Pentron Clinical of Orange, Calif. which use fiberglass within a mixture of cured copolymers bisphenol A-glycidyl methacrylate (BISGMA), urethane dimethacrylate (UDMA) and HDDMA. The Safety Data Sheet for Pentron lists 1,6-hexanediyl bismethacrylate from 10-30%, 7,7,9(or 7,9,9)-trimethyl-4,13-dioxo-3,14-dioxa-5,12-diazahexadecane-1,16-diylbismethacrylate from 5-10%, diphenyl(2,4,6-trimethylbenzoyl)phosphine oxide from 0.1 to 1% and 2-(Diethylamino)ethyl methacrylate from 0.1 to 1%.

The fibers of FiberKleer posts may be barium borosilicate glass, glass fibers. In this invention, quartz fibers may be used alone or in addition to other dentally acceptable fibers. Other acceptable glass fibers include Reforpost Glass Fibers from ANGELUS INDÚSTRIA DE PRODUTOS ODONTOLÓGICOS S/A sociedad anonima (sa) BRAZIL Rua Waldir Landgraf, 101 Lindóia, Londrina, PR, BRAZIL which makes glass fiber infraradicular posts with conical tips. ParaPost® Fiber Lux from Coltène/Whaledent Inc. of Cuyahoga falls, Ohio also makes an acceptable glass-filled composite.

The fibers are embedded within dentally acceptable composite resins, also already used in dentistry for decades, to form the dental crowns according to standard procedures for creating dental crowns. The outer surface of the dental crowns is a cosmetic resin composite substrate which can provide the appropriate color and good mouth feel.

Fiber-reinforced composites for dental materials are discussed in U.S. Pat. No. 7,673,550 to Karmaker et al., the disclosure of which is incorporated herein by reference. Contrary to that patent, this invention does not contemplate the need to form rods or sheaths and instead forms the dental crowns from a mixture of fiberglass/quartz fibers and resin without orienting into solid rods.

The methods includes roughening the outer surface of the tooth (teeth) to be restored, applying a bonding agent in the crown of the invention to be seated on the tooth/teeth to be restored tooth with then mechanical/chemical retention.

The dental crowns may include from 25 to 85% fibers and more preferably between 30 to 70%, with the remainder being the resin and fillers. The outer layer is a cosmetic resin composite bonded to the main body of the dental crown which is formed from the fiber/resin mixture.

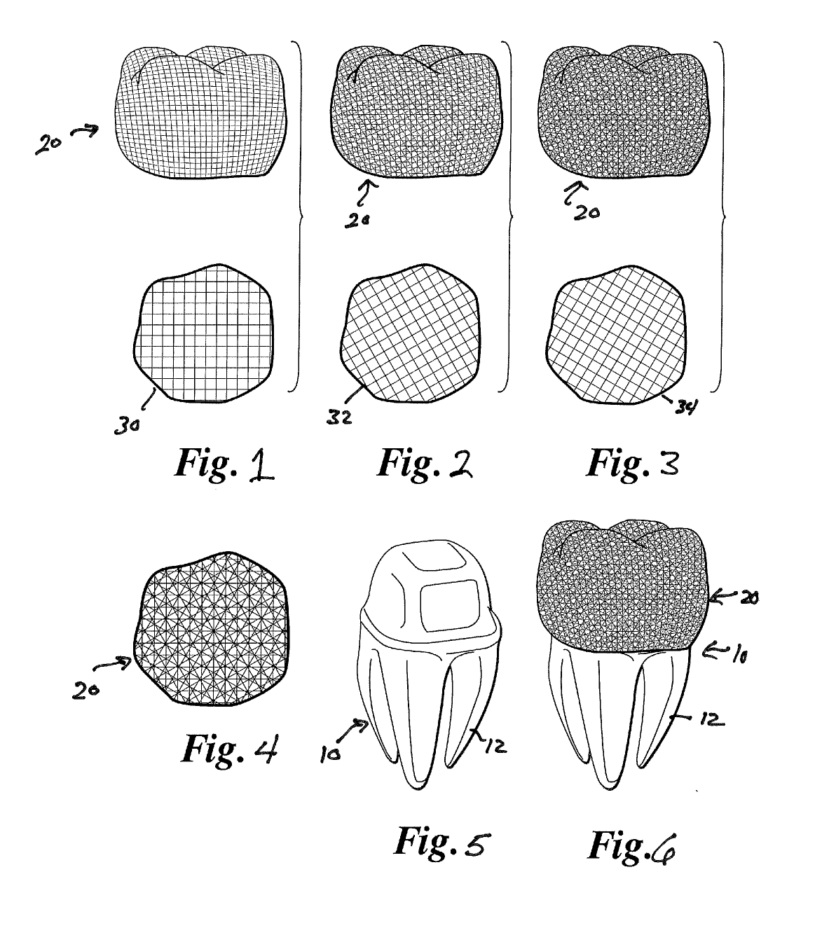

As shown in FIGS. 1-4, the dental crown 20 of the invention is formed of layers of fiberglass mesh sheets 30 from one to preferably three sheets. More sheets may be used up until their combined thickness makes it unneeded. Generally, more than 6 sheets may be difficult unless the sheet thickness is smaller. The fiberglass sheets are preferably about 0.0035 inches (0.0889 mm) thick with sheet weights of about 2.4 ounces per square yard (8.1 grams per square millimeter. The charts below show aspects of the invention with SA referencing Surface Area, V for Volume and Tooth Type referring to typical teeth sizes to note relative sizes.

Note that if more than one mesh sheet 30 is used each successive sheet is preferably rotated form the first sheet as shown in FIGS. 1-4. The second mesh sheet 32 is shown in FIG. 2, the third sheet 34 is shown in FIG. 3. The effect of all three mesh sheets 30, 32 and 34 is shown in FIG. 4 which shows how coverage over the dental crown 20 is excellent with additional mesh sheets. Mesh sheets 30, 32 and 34 may all be identical other than their rotation relative to another layer. Each of FIGS. 1-3 show a top view of the newly added mesh sheet as well as a perspective view of the cumulative effect of adding another layer to the previous figure.

A mold (not shown) is used for each dental crown to be formed and the fibers are layered, preferably in different directions and the mold is closed and resin is injected into the mold under pressure to embed the fibers and provide great strength. The fiberglass layers may be in a mat but surprisingly, individual layers of fiberglass sheets provided better strength at a better cost point. The fiberglass is saturated with the resin through the dental crown and is trimmed as need after removed from the mold.

The resin may be a suitable dental resin as used in the industry. It may be an epoxy resin such as EPO-TEK 301 from Epoxy Technology, Inc., 14 Fortune Drive, Billerica, Mass. 01821 USA. Its EPO-TEK 301, as with all epoxies, is a two component epoxy with Part A and Part B. Part A contains a Bisphenol A Diglycidyl Ether Resin and a reactive diluent. Part B contains trimethyl-1,6-Hexanediamine. Any dentally accepted resin including epoxies may be used, the Epo-Tek 301 has been tested and works well.

Dentistry involves working with patients that have a wide range of tooth colors. A colorant may be added to the resin to produce the color that best matches the tooth which will receive a crown. Dental colorants are well known. A suitable and typical colorant is titanium oxide, TiO₂. Titanium oxide is typically from 0.3 to 1.2 micrometers in size. Iron oxide (FeO₂) may be used to impart a yellowing color.

Total SA and V

| Tooth Type | SA (mm\hat{ }2) | Volume (mm\hat{ }3) |
|---|---|---|
| A medium | 218.12 | 239.932 |
| B small | 164.48 | 180.928 |
| C medium | 120.86 | 132.946 |
| E small | 98.65 | 108.515 |

Fiber Glass

| | in | mm | | |
|---|---|---|---|---|
| Thickness | 0.0035 | 0.0889 | | |
| weight (sheet) | 2.4 | oz/yd\hat{ }2 | 8.1374E−05 | g/mm\hat{ }2 |

Epo Tek 301

| mix ratio by | | | 5 total | |
|---|---|---|---|---|
| weight | 20:5 ratio | 4:1 ratio | parts | |
| cured density | 1.08 | g/cm\hat{ }3 | 0.00108 | g/(mm\hat{ }3) |

TiO₂

| | |
|---|---|
| mix ratio by | 5:0.015 |
| weight with epo | ratio |
| tek 301 | |

| | A size medium | | |
|---|---|---|---|
| | Ratios of products by mass | | |
| | (grams) | | |
| | Epo Tek | Fiber | |
| | 301 | Glass | TiO₂ |
| as tested | 0.259127 | 0.0887462 | 0.000778 |
| low | 0.064782 | 0.0221865 | 0.000194 |
| high | 0.518253 | 0.1774924 | 0.001556 |

| | B size small | | |
|---|---|---|---|
| | Ratios of products by mass | | |
| | (grams) | | |
| | Epo Tek | Fiber | |
| | 301 | Glass | TiO₂ |
| as | 0.195402 | 0.066922 | 0.000586 |
| tested | | | |
| low | 0.048851 | 0.01673 | 0.000147 |
| high | 0.390804 | 0.133844 | 0.001172 |

| | C size medium | | |
|---|---|---|---|
| | Ratios of products by mass | | |
| | (grams) | | |
| | Epo Tek | Fiber | |
| | 301 | Glass | TiO₂ |
| as tested | 0.143582 | 0.0491741 | 0.000431 |
| low | 0.035895 | 0.0122935 | 0.000108 |
| high | 0.287163 | 0.0983483 | 0.000861 |

| | C size medium | | |
|---|---|---|---|
| | Ratios of products by mass | | |
| | (grams) | | |
| | Epo Tek | Fiber | |
| | 301 | Glass | TiO₂ |
| as | 0.117196 | 0.040138 | 0.000352 |
| tested | | | |
| low | 0.029299 | 0.010034 | 8.79E−05 |
| high | 0.234392 | 0.080275 | 0.000703 |

| | Overall Mix ratios by mass | | |
|---|---|---|---|
| | Epo Tek | Fiber Glass | TiO₂ |
| As tested | 33.22135 | 11.377716 | 1 |
| 75% | 8.305338 | 2.8444291 | 0.25 |
| reduction | | | |
| 75% increase | 58.13737 | 19.911004 | 1.75 |

multiplier for ratio calculation: 128.2051282
Final ratio: 8-58 parts Epotek 2.8-20 parts Fiber Glass and 0.25-1.75 parts TiO₂

Testing of sample dental crowns made without fiberglass or TiO₂ were conducted and the average force before breaking was 27.9 pounds. A stainless steel dental crown test failed with ductile fracturing at 160 pounds. A zirconia dental crown test failed with a brittle fracture at only 75 pounds. Failed zirconia crowns are brittle and sharp shards are created which is a problem. A dental crown of the invention made with epoxy and fiberglass didn't fail until a force of 199 pounds was applied and then with a plastic deformation. Thus, the fiberglass dental crowns of the invention absorbed 2 to 2.5 times the force of the zirconia crowns. The fiberglass dental crowns of the invention are therefore safer and less hazardous than zirconia crowns. Human bite strength on chewing yields about 72 pounds of force which is close to the failure point for zirconia and well below the failure point of the inventive crowns.

Fibers have referenced fiberglass but other fibers may be used including quartz fibers, carbon fibers and aramid fibers such as DuPont Kevlar® brand fibers. Use of the terms “fibers” and “fiberglass” herein are intended to encompass a wide range of fibers that may be woven into mesh sheets that will impart strength into a dental crown of the invention.

Dental crowns are primarily used in humans but may be used in veterinary applications as well.

While this invention may be embodied in many different forms, there are shown in the drawings and described in detail herein specific preferred embodiments of the invention. The present disclosure is an exemplification of the principles of the invention and is not intended to limit the invention to the particular embodiments illustrated.

This completes the description of the preferred and alternate embodiments of the invention. Those skilled in the art may recognize other equivalents to the specific embodiment described herein which equivalents are intended to be encompassed by the claims attached hereto.

1. A dental crown in which the crown is substantially formed from dentally acceptable resins which encase at least one mesh fiber sheet therewithin.

2. The dental crown of claim 1 in which the fiber of the mesh fiber is selected from the group consisting of fiberglass, quartz fiber, aramid fiber and carbon fibers.

3. The dental crown of claim 2 including three mesh fiber sheets, each of which is rotationally offset from the other of said three sheets.

4. The dental crown of claim 1 wherein said dentally acceptable resin is an epoxy resin.

5. The dental crown of claim 1 wherein said mesh fiber sheet is formed of fiberglass fibers.

6. The dental crown of claim 1 further including a colorant.

7. The dental crown of claim 6 wherein said colorant is titanium oxide.

8. A dental crown comprising a dental epoxy, fiberglass in the form of at least one mesh fiber sheet and a colorant.

9. The dental crown of claim 8 wherein said dental epoxy comprises about 8 to about 58 parts, the fiberglass comprises about 2.8 to about 20 parts and the colorant comprises about 0.25 to about 1.75 parts.

10. The dental crown of claim 9 including three mesh fiber sheets, each of which is rotationally offset from the other of said three sheets.

11. A dental crown comprising a dental epoxy, at least one mesh fiber sheet embedded within said epoxy and a colorant, said at least one mesh fiber sheet being formed from a fiberglass, carbon fiber, aramid fiber or quartz fiber.

12. The dental crown of claim 11 including three mesh fiber sheets, each of which is rotationally offset from the other of said three sheets.

13. The dental crown of claim 12 wherein said dental epoxy comprises about 8 to about 58 parts, the fiberglass comprises about 2.8 to about 20 parts and the colorant comprises about 0.25 to about 1.75 parts.

14. The dental crown of claim 13 further including a colorant.
